(19) United States
(12) Patent Application Publication
(10) Pub. No.: US 2025/0179579 A1
(43) Pub. Date: Jun. 5, 2025

(54) METHOD OF DETECTING UROTHELIAL OR BLADDER CANCER IN A LIQUID SAMPLE

(71) Applicants: QIAGEN GMBH, Hilden (DE); STRATIFYER MOLECULAR PATHOLOGY GMBH, Köln (DE)

(72) Inventors: Ronny KELLNER, Hilden (DE); Ralph WIRTZ, Köln (DE); Thorsten ECKE, Bad Saarow (DE)

(21) Appl. No.: 18/724,782

(22) PCT Filed: Dec. 27, 2022
(86) PCT No.: PCT/EP2022/087921

§ 371 Date: Jun. 27, 2024

(30) Foreign Application Priority Data
Dec. 27, 2021 (EP) 21217884.2

(51) Int. Cl. C12Q 1/6886; C12Q 1/686

(52) U.S. Cl. CPC C12Q 1/6886; C12Q 1/686; C12Q 2600/112; C12Q 2600/158

(57) ABSTRACT

The present disclosure relates to a method of classifying a patient that suffers from or is at risk of developing urothelial or bladder cancer. The method involves the steps of taking a liquid sample from the patient, extracting RNA from the liquid sample, and determining in the RNA, the expression level of at least one gene encoding for a receptor selected from the group consisting of FGFR2, FGFR3 or ErbB2, and classifying the sample of the patient into one of at least two classifications.

### INTRODUCTION

The present application relates to the field of molecular diagnostics.

Urothelial cancer (UC) is one of the 10 most common malignancies worldwide with nearly 386,000 new cases and nearly 150,200 deaths per year, characterized by high rates of recurrence and progression. For decades, the only therapy regimen for metastatic UC was platinum-based chemotherapy, which is accompanied with a poor 5-year overall survival of ≤ 15% and a very poor prognosis for patients who fail the standard chemotherapy regimen.

Similar findings apply to bladder cancer (BC) with an estimated 549,393 new cases occurring in 2018 alone on the global scale. Both non-muscle-invasive bladder cancer (NMIBC) and muscle-invasive bladder cancer (MIBC) show high recurrence and progression rates, and therefore, imply a great burden being placed on patients and health care systems worldwide. In around ¾ of patients, a NMIBC is diagnosed, and within the remaining quarter, a metastatic stage is found in 5% of cases.

Today's therapy strategies are mainly based on histopathological tumor stage and grading with local management in NMIBC and systematic treatment and radical cystectomy in MIBC.

However, even after radical therapy, local recurrences and distant metastasis occur in up to 50% of cases. However, the determination of histological grade suffers tremendous inter- and intraobserver variabilities. The general conformity in staging and grading is between 50% and 60%.

However it is obvious that sensitive detection of bladder cancer and urothelial cancer both for screening and monitoring of recurrences would be desirable for curative approaches and improved treatment decisions.

Early detection of tumor recurrences and objective assessment of tumor grade or subtype by a non-invasive method could spare unnecessary biopsies and thereby reduce costs and improve quality of life of bladder cancer patients.

### SUMMARY

In an embodiment, a method of classifying a patient that suffers from, or is at risk of developing, urothelial or bladder cancer is provided. The method may involve a multitude of steps. A first step involves taking a urine sample from the patient. A second step involves extracting RNA from the urine sample. A third step involves determining in the RNA, the expression level of one or more gene encoding for a receptor selected from the group consisting of FGFR2, FGFR3, or ErbB2. And a fourth step involves classifying the urine sample of the patient from the outcome of the third step into one of at least two classifications. Further, the one or more expression levels determined in the third step are normalized with the expression level of housekeeping gene CALM2 in order to obtain one or more normalized expression levels.

### BRIEF DESCRIPTION OF THE FIGURES

FIG. 1: Spearman correlation of relative gene expression determined by dPCR on QIAcuity for ERBB2, FGFR2 and FGFR3 from urine exosomal RNA samples and matched FFPE tissues from bladder biopsies. “c” indicates gene expression values from urine after CALM2 normalization. “a” indicates gene expression values from urine after ACTB normalization.

FIG. 2: Spearman correlation of relative gene expression determined by dPCR on QIAcuity for ERBB2, FGFR2 and FGFR3 from urine with tumor stage. “c” indicates gene expression values from urine after CALM2 normalization. “a” indicates gene expression values from urine after ACTB normalization. For the tumor stage, score values have been assigned to the different tumor stages, as described below.

FIG. 3: Spearman correlation of relative gene expression determined by dPCR on QIAcuity for ERBB2, FGFR2 and FGFR3 from urine with tumor grade. “c” indicates gene expression values from urine after CALM2 normalization. “a” indicates gene expression values from urine after ACTB normalization. For the tumor grade, the WHO 1973 classification has been used, as described below.

### DETAILED DESCRIPTION

Before the invention is described in detail, it is to be understood that this invention is not limited to the particular component parts of the devices described or process steps of the methods described as such devices and methods may vary. It is also to be understood that the terminology used herein is for purposes of describing particular embodiments only, and is not intended to be limiting. It must be noted that, as used in the specification and the appended claims, the singular forms “a”, “an”, and “the” include singular and/or plural referents unless the context clearly dictates otherwise. It is moreover to be understood that, in case parameter ranges are given which are delimited by numeric values, the ranges are deemed to include these limitation values.

It is further to be understood that embodiments disclosed herein are not meant to be understood as individual embodiments which would not relate to one another. Features discussed with one embodiment are meant to be disclosed also in connection with other embodiments shown herein. If, in one case, a specific feature is not disclosed with one embodiment, but with another, the skilled person would understand that does not necessarily mean that said feature is not meant to be disclosed with said other embodiment. The skilled person would understand that it is the gist of this application to disclose said feature also for the other embodiment, but that just for purposes of clarity and to keep the specification in a manageable volume this has not been done.

Furthermore, the content of the prior art documents referred to herein is incorporated by reference. This refers, particularly, for prior art documents that disclose standard or routine methods. In that case, the incorporation by reference has mainly the purpose to provide sufficient enabling disclosure, and avoid lengthy repetitions.

According to a first aspect of the disclosure, a method of classifying a patient that suffers from or is at risk of developing urothelial or bladder cancer, is provided, said method comprising the steps of:
a) taking a liquid sample from the patient,
b) extracting RNA from said liquid sample, and
c) determining in said RNA, the expression level of at least one gene encoding for a receptor selected from the group consisting of FGFR2, FGFR3 or ErbB2
d) classifying the sample of said patient from the outcome of step c) into one of at least two classifications.

In one embodiment, the patient suffers from or is at risk of developing bladder cancer.

Fibroblast growth factor receptors (FGFR) are, as their name implies, receptors that bind to members of the fibroblast growth factor family of proteins. The fibroblast growth factor receptors consist of an extracellular ligand domain composed of three immunoglobulin-like domains, a single transmembrane helix domain, and an intracellular domain with tyrosine kinase activity. These receptors bind fibroblast growth factors, members of the largest family of growth factor ligands, comprising 22 members.

FGFRs are receptor tyrosine kinases of ˜800 amino acids with several domains including three extracellular immunoglobulin-like domains (D1-D3), a transmembrane domain (TM), and two intracellular tyrosine kinase domains (TK1 and TK2).

The natural alternate splicing of four fibroblast growth factor receptor (FGFR) genes results in the production of over 48 different isoforms of FGFR. These isoforms vary in their ligand-binding properties and kinase domains.

The three immunoglobin (Ig)-like domains present a stretch of acidic amino acids (“the acid box”) between D1 and D2. This “acid box” can participate in the regulation of FGF binding to the FGFR. Immunoglobulin-like domains D2 and D3 are sufficient for FGF binding. Each receptor can be activated by several FGFs. In many cases, the FGFs themselves can also activate more than one receptor (i.e., FGF1, which binds all seven principal FGFRs). FGF7, however, can only activate FGFR2 and FGF18 was recently shown to activate FGFR3.

Receptor tyrosine-protein kinase ErbB-2, also known as CD340 (cluster of differentiation 340), proto-oncogene Neu, Erbb2 (rodent), or ERBB2 (human), is a protein that in humans is encoded by the ERBB2 gene. ERBB is abbreviated from erythroblastic oncogene B, a gene isolated from avian genome. It is also frequently called HER2 (from human epidermal growth factor receptor 2) or HER2/neu. HER2 is a member of the human epidermal growth factor receptor (HER/EGFR/ERBB) family. Amplification or over-expression of this oncogene has been shown to play an important role in the development and progression of certain aggressive types of breast cancer. In recent years the protein has become an important biomarker and target of therapy for approximately 30% of breast cancer patients.

The relationship between bladder cancer and urothelial cancer and the expression of FGFR genes is disclosed in WO2020208260A1, the content of which is incorporated herein by reference. The inventors have for the first time shown that a liquid sample can be used for the determination of the respective expression levels, without losing preciseness and specificity. This is a tremendous progress in terms of patient compliance and ease of sample taking, which reduces pain and discomfort in the patient, and allows determination of the most promising therapy option, or avoidance of non-promising therapy options. Furthermore, this provides the possibility to take samples at very early stages, so as to detect malignancies, or predict upcoming malignancies, at a point of time where treatment options are still manifold.

The following table provides information with regard to the three genes, together with the respective mRNA sequences have been identified in humans. It should be noted that the skilled person is capable of selecting suitable primer combinations (with optionally a probe) to identify and quantify the expression of any of these genes on the basis of the disclosure provided herein combined with his routine knowledge.

TABLE 1
Details of FGFR2, FGFR 3 and ErbB2

| | | | mRNA NCBI Reference Sequence |
|---|---|---|---|
| | | | (examples, other isoforms or |
| | | | variants may exist and can |
| | | Entrez | easily be found by the skilled |
| Gene | Alias | Gene ID | person in the respective databases) |
| FGFR2 | CD332 | 2263 | NM_000141 |
| | | | NM_001144913 |
| | | | NM_001144914 |
| | | | NM_001144915 |
| | | | NM_001144916 |
| FGFR3 | CD333 | 2261 | NM_000142 |
| | | | NM_001163213 |
| | | | NM_022965 |
| | | | NM_001354809 |
| | | | NM_001354810 |
| ErbB2 | Her2/neu | 2064 | NM_001005862 |

Generally, the terms “urothelial cancer” and “bladder cancer” have overlapping scope and are sometimes being used interchangeably. Sometimes, the term “urothelial cancer” is used as a generic definition, and “bladder cancer” is used to determine a given species of urothelial cancer. Sometimes, the term “urothelial cancer” is used to designate cancer in the urether, while “bladder cancer” is used designate cancer in the bladder as such.

According to one embodiment, the RNA is extracted from exosomes in the liquid sample.

Exosomes are tiny vesicles (30-150 nm) constantly produced by both healthy and abnormal cells, and found in all body fluids. These vesicles, loaded with RNA and protein cargo, have a wide range of biological functions, including cell-to-cell communication and signalling. Exosomes can be isolated and analyzed for the RNA content, with methods described herein elsewhere.

Kits for isolating and/or analyzing exosomal RNA are for example provided by QIAGEN (Hilden, Germany) (exoEasy Kit, miRCURY Exosome Kits).

By binding water molecules, some exosomal RNA isolation kits for less soluble components such as vesicles out of solution, allowing them to be collected by a short centrifugation at low speed. The reagent is added to the sample and the solution is incubated at room temperature for 1 hour. The precipitated exosomes are collected by standard centrifugation at 10,000×g for 1 hour at 2-8° C. The pellet is then resuspended in PBS or similar buffer, and the exosomes are ready for subsequent analysis or further purification by affinity methods. Total RNA and protein can then be purified.

Another option is to use targeted immunomagnetic beads coated with any one of CD9, CD63, CD81.

Still another approach is to use a membrane-based affinity binding step to isolate exosomes liquid samples. The method does not distinguish exosomes by size or cellular origin, and is not dependent on the presence of a particular epitope. Instead, it makes use of a generic, biochemical feature of vesicles to recover the entire spectrum of extracellular vesicles present in a sample. The approach uses a spin column format and specialized buffers to purify exosomes from pre-filtered biological fluids.

These and other methods to isolate exosomal RNA are discussed in Li et al. 2017, the content of which is incorporated herein by references for enablement purposes.

The inventors have shown, for the first time, that exosomes can be used to determine the expression levels of FGFR2, FGFR3 and ErbB2, and provide a meaningful analysis, including prediction of therapy success, with regard to bladder cancer and/or urothelial cancer.

According to one embodiment, in step c) the expression level of at least ErbB2 is determined.

According to one embodiment, in step c) the expression level of at least
ErbB2 and
FGFR2 and/or FGFR3
   is determined.

According to one embodiment, the liquid sample is at least one selected from the group consisting of
urine
blood
blood serum or plasma
saliva, and/or
lymph liquid

In one embodiment, the sample is a urine sample. In one embodiment, the sample has been taken before a bladder tumor has been removed. In one embodiment, the sample is a pre-TURB urine sample. The term “TURB”, as used herein, relates to the transurethral resection of the bladder. In such surgery, a bladder tumor is removed via the urethra with the help of a resectoscope.

According to one embodiment, said expression level(s) is/are determined by at least one of
(i) a hybridization based method, in which labeled, single stranded probes are used
(ii) a PCR based method, which method comprises a polymerase chain reaction (PCR)
(iii) a method based on the electrochemical detection of particular molecules, which method encompasses an electrode system to which molecules bind under creation of a detectable signal,
(iv) an array based method, which comprises the use of a m microarray and/or biochip, and/or
(v) an immunological method, in which one or more target-specific protein binders are used.

The term “a PCR based method” as used herein refers to methods comprising a polymerase chain reaction (PCR). This is an approach for exponentially amplifying nucleic acids, like DNA or RNA, via enzymatic replication, without using a living organism. As PCR is an in vitro technique, it can be performed without restrictions on the form of DNA, and it can be extensively modified to perform a wide array of genetic manipulations. When it comes to the determination of expression levels, a PCR based method may for example be used to detect the presence of a given mRNA by (1) reverse transcription of the complete mRNA pool (the so called transcriptome) into cDNA with help of a reverse transcriptase enzyme, and (2) detecting the presence of a given cDNA with help of respective primers. PCR-based methods comprise in particular quantitative PCR (qPCR) and digital PCR (dPCR).

The term “Quantitative real-time PCR” (qPCR)”—sometimes also called real time PCR (RT PCR) refers to any type of a PCR method which allows the quantification of the template in a sample. Quantitative real-time PCR comprise different techniques of performance or product detection as for example the TaqMan technique or the LightCycler technique. The TaqMan technique, for examples, uses a dual-labelled fluorogenic probe. The TaqMan real-time PCR measures accumulation of a product via the fluorophore during the exponential stages of the PCR, rather than at the end point as in conventional PCR. The exponential increase of the product is used to determine the threshold cycle, CT, i.e. the number of PCR cycles at which a significant exponential increase in fluorescence is detected, and which is directly correlated with the number of copies of DNA template present in the reaction. The set up of the reaction is very similar to a conventional PCR, but is carried out in a real-time thermal cycler that allows measurement of fluorescent molecules in the PCR tubes. Different from regular PCR, in TaqMan real-time PCR a probe is added to the reaction, i.e., a single-stranded oligonucleotide complementary to a segment of 20-60 nucleotides within the DNA template and located between the two primers. A fluorescent reporter or fluorophore (e.g., 6-carboxyfluorescein, acronym: FAM, or tetrachlorofluorescin, acronym: TET) and quencher (e.g., tetramethylrhodamine, acronym: TAMRA, of dihydrocyclopyrroloindole tripeptide “minor groove binder”, acronym: MGB) are covalently attached to the 5′ and 3′ ends of the probe, respectively. The close proximity between fluorophore and quencher attached to the probe inhibits fluorescence from the fluorophore. During PCR, as DNA synthesis commences, the 5′ to 3′ exonuclease activity of the Taq polymerase degrades that proportion of the probe that has annealed to the template (Hence its name: Taq polymerase+PacMan). Degradation of the probe releases the fluorophore from it and breaks the close proximity to the quencher, thus relieving the quenching effect and allowing fluorescence of the fluorophore. Hence, fluorescence detected in the realtime PCR thermal cycler is directly proportional to the fluorophore released and the amount of DNA template present in the PCR.

Digital PCR is a biotechnological refinement of conventional polymerase chain reaction methods that can be used to directly quantify and clonally amplify nucleic acids strands including DNA, cDNA, or RNA (the latter has to be revers transcribed into cDNA). The key difference between dPCR and traditional PCR lies in the method of measuring nucleic acids amounts, with the former being a more precise method than PCR, though also more prone to error in the hands of inexperienced users. A “digital” measurement quantitatively and discretely measures a certain variable, whereas an “analog” measurement extrapolates certain measurements based on measured patterns. PCR carries out one reaction per single sample. dPCR also carries out a single reaction within a sample, however the sample is separated into a large number of partitions and the reaction is carried out in each partition individually. This separation allows a more reliable collection and sensitive measurement of nucleic acid amounts. The method has been demonstrated as useful for studying variations in gene sequences-such as copy number variants and point mutations

In one embodiment, said dPCR is carried out on the QIAcuity Digital PCR System provided by QIAGEN (Hilden, Germany).

A “microarray” herein also refers to a “biochip” or “biological chip”. an array of regions having a density of discrete regions of at least about 100/cm 2, and preferably at least about 1000/cm². The regions in a microarray have typical dimensions, e.g., diameters, in the range of between about 10-250 μm, and are separated from other regions in the array by about the same distance.

The term “hybridization-based method”, as used herein, refers to methods imparting a process of combining complementary, single-stranded nucleic acids or nucleotide analogues into a single double stranded molecule. Nucleotides or nucleotide analogues will bind to their complement under normal conditions, so two perfectly complementary strands will bind to each other readily. In bioanalytics, very often labeled, single stranded probes are in order to find complementary target sequences. If such sequences exist in the sample, the probes will hybridize to said sequences which can then be detected due to the label. Other hybridization based methods comprise microarray and/or biochip methods. Therein, probes are immobilized on a solid phase, which is then exposed to a sample. If complementary nucleic acids exist in the sample, these will hybridize to the probes and can thus be detected. These approaches are also known as “array based methods”. Yet another hybridization based method is PCR, which is described above. When it comes to the determination of expression levels, hybridization based methods may for example be used to determine the amount of mRNA for a given gene.

The term “method based on the electrochemical detection of molecules” relates to methods which make use of an electrode system to which molecules, particularly biomolecules like proteins, nucleic acids, antigens, antibodies and the like, bind under creation of a detectable signal. Such methods are for example disclosed in WO0242759, WO0241992 and WO02097413 filed by the applicant of the present disclosure, the content of which is incorporated by reference herein. These detectors comprise a substrate with a planar surface which is formed, for example, by the crystallographic surface of a silicon chip, and electrical detectors which may adopt, for example, the shape of interdigital electrodes or a two dimensional electrode array. These electrodes carry probe molecules, e.g. nucleic acid probes, capable of binding specifically to target molecules, e.g. target nucleic acid molecules. The probe molecules are for example immobilized by a Thiol-Gold-binding. For this purpose, the probe is modified at its 5′- or 3′-end with a thiol group which binds to the electrode comprising a gold surface. These target nucleic acid molecules may carry, for example, an enzyme label, like horseradish peroxidise (HRP) or alkaline phosphatase. After the target molecules have bound to the probes, a substrate is then added (e.g., α-naphthyl phosphate or 3,3′5,5′-tetramethylbenzidine which is converted by said enzyme, particularly in a redox-reaction. The product of said reaction, or a current generated in said reaction due to an exchange of electrons, can then be detected with help of the electrical detector in a site specific manner.

According to one embodiment, the one or more expression level(s) determined in step a) are normalized with one or more expression level(s) of one or more reference genes to obtain one or more normalized expression level(s).

Reference genes in PCR are discussed in Kozera and Rapacz (2013), the content of which is incorporated herein by reference.

In order to normalize the expression level of a given gene, a comparison to a reference gene is preferably made. In a RT PCR method, the normalized gene expression of a target gene is calculated by the following formula:

$$40 - ( ( Ct target gen ) - ( Ct reference gene ) )$$

also called “ΔCT” herein.

In a dPCR based method, the copy numbers of the target gene as determined by dPCR (e.g., as cDNA copy numbers per μl of sample) are normalized by division with the respective copy numbers of the reference gene. The resulting value is dimensionless.

According to one embodiment, said one or more reference gene(s) is at least one housekeeping gene.

The term “housekeeping gene”, as used herein, refers to a more specialized form of a reference gene. It refers to a group of genes that codes for proteins whose activities are essential for the maintenance of cell function. These genes are typically similarly expressed in all cell types.

Housekeeping genes include, without limitation, glyceraldehyde-3-phosphate dehydrogenase (GAPDH), Cypl, albumin, actins, e.g. β-actin, tubulins, cyclophilin, hypoxantine phsophoribosyltransferase (HRPT), L32. 28S, and 18S.

According to one embodiment, the at least one housekeeping gene is selected from the group consisting of ACTB, CALM2, B2M and/or RPL37A, as shown in the following table.

It should be noted that the skilled person is capable of selecting suitable primer combinations (with optionally a probe) to identify and quantify the expression of any of these genes on the basis of the disclosure provided herein combined with his routine knowledge.

TABLE 2
Details of housekeeping genes

| | | Entrez | mRNA NCBI Reference |
|---|---|---|---|
| Gene | Alias | Gene ID | Sequence (examples) |
| ACTB | Beta-actin | 60 | NM_001101 |
| CALM2 | Calmodulin 2 | 805 | NM_001305624 |
| | | | NM_001305625 |
| | | | NM_001305626 |
| | | | NM_001743 |
| B2M | β-2 | 567 | NM_004048.3 |
| | microglobulin | | |
| RPL37A | ribosomal | 6168 | NM_000998 |
| | protein L37a | | |

Among these, in one embodiment, at least one housekeeping gene is CALM2.

According to one embodiment, the step d) of classifying the sample of said patient from the outcome of step c) into one of at least two classifications comprises a classification into at least one of
urothelial or bladder cancer negative
urothelial or bladder cancer positive
low risk urothelial or bladder cancer
high risk urothelial or bladder cancer

In the following table, these possible classifications are shown together with possible or recommendable therapeutic options

TABLE 3
Classifications vs possible therapeutic options

| | Expression | | Possible or recommendable |
|---|---|---|---|
| Gene | status | classification | therapeutic options |
| ErbB2 | High or | urothelial or bladder | Surgery |
| | increased | cancer positive OR | intravesical instillation therapy |
| | | High Risk | (BCG, Mitomycn, Gemticabin) |
| | | | chemotherapy, anti-Her2 therapy) |
| | | | systemic therapy (chemotherapy |
| | | | with e.g. Cisplatin/GC or MVAC, |
| | | | anti-Her2 therapy) |
| ErbB2 | Low or not | urothelial or bladder | Monitoring |
| | increased | cancer negative OR | Tretment with Trastuzumab |
| | | Low Risk | deruxtecan (anti-Her2 antibody |
| | | | drug conjugate indicated for |
| | | | tumors with low or heterogeneous |
| | | | expression of ErbB2 |
| FGFR2/3 | High or | Low risk urothelial or | Treatment with FGFR inhibitors |
| | increased | bladder cancer | |

In a set of embodiments, the expression level of one of the above genes is determined as “high” or “low” by digital PCR (dPCR) (given in cDNA copy numbers×μl⁻¹, normalized by the respective copy numbers of a housekeeper (in this case CALM2 or ACTB).

For this purpose, the following thresholds (copy numbers×μl⁻¹) can be used:
ErbB2: high expression: ≥0.1; ≥0.15; ≥0.2; ≥0.25; ≥0.3; ≥0.35; ≥0.4; ≥0.45; ≥0.5; ≥0.55; ≥ 0.6; ≥0.65; ≥0.7; ≥0.75; ≥0.8; ≥0.85; ≥0.9; ≥0.95; ≥1; ≥1.05; ≥1.1; ≥1.15; ≥1.2; ≥1.25; ≥ 1.3; ≥1.35; ≥1.4; ≥1.45; or ≥1.5;
FGFR2: high expression: ≥0.001; ≥0.002; ≥0.003; ≥0.004; ≥0.005; ≥0.006; ≥0.007; ≥0.008; ≥0.009; ≥0.01; ≥0.011; ≥0.012; ≥0.013; ≥0.014; ≥0.015; ≥0.016; ≥0.017; ≥0.018; ≥0.019; ≥0.02; ≥0.021; ≥0.022; ≥0.023; ≥0.024; ≥0.025; ≥0.026; ≥0.027; ≥0.028; ≥0.029; or ≥0.03
FGFR3: high expression: ≥0.001; ≥0.002; ≥0.003; ≥0.004; ≥0.005; ≥0.006; ≥0.007; ≥0.008; ≥0.009; ≥0.01; ≥0.011; ≥0.012; ≥0.013; ≥0.014; ≥0.015; ≥0.016; ≥0.017; ≥0.018; ≥0.019; ≥0.02; ≥0.021; ≥0.022; ≥0.023; ≥0.024; ≥0.025; ≥0.026; ≥0.027; ≥0.028; ≥0.029; or ≥0.03

In a set of embodiments, the expression level of one of the above genes is determined as “high” or “low” by digital PCR (dPCR) (given in cDNA copy numbers×μl⁻¹, normalized by the respective copy numbers of a housekeeper (in this case CALM2 or ACTB).

For this purpose, the following thresholds (CT values) can be used:
ErbB2: high expression: ≤42.50; ≤41.62; ≤40.99; ≤40.51; ≤40.11; ≤39.78; ≤39.49; ≤39.23; ≤39.01; ≤38.80; ≤38.61; ≤38.44; ≤38.27; ≤38.12; ≤37.98; ≤37.85; ≤37.73; ≤37.61; ≤37.50; ≤37.39; ≤37.29; ≤37.20; ≤37.10; ≤37.02; ≤36.93; ≤36.85; ≤36.77; ≤36.69; or ≤36.62.
FGFR2: high expression: ≤52.50; ≤50.99; ≤50.11; ≤49.49; ≤49.01; ≤48.61; ≤48.27; ≤47.98; ≤47.73; ≤47.50; ≤47.29; ≤47.10; ≤46.93; ≤46.77; ≤46.62; ≤46.48; ≤46.35; ≤46.22; ≤46.11; ≤45.99; ≤45.89; ≤45.79; ≤45.69; ≤45.60; ≤45.51; ≤45.43; ≤45.34; ≤45.26; ≤45.19; or ≤ 45.11.
FGFR3: high expression: ≤52.50; ≤50.99; ≤50.11; ≤49.49; ≤49.01; ≤48.61; ≤48.27; ≤47.98; ≤47.73; ≤47.50; ≤47.29; ≤47.10; ≤46.93; ≤46.77; ≤46.62; ≤46.48; ≤46.35; ≤46.22; ≤46.11; ≤45.99; ≤45.89; ≤45.79; ≤45.69; ≤45.60; ≤45.51; ≤45.43; ≤45.34; ≤45.26; ≤45.19; or ≤ 45.11.

In a set of embodiments, the expression status of one of the above genes is determined as “increased” or “not increased” by comparing, in a given patient, the actual expression, as determined either by digital PCR (dPCR) or real time PCR (rtPCR), with a basal expression levels (“baseline”) of the same patient.

Such basal expression level can for example be determined by three measurements taken with an interval for four weeks each, and determining the average thereof.

Based on such method, the expression level of ErbB2, FGFR2 and/or FGFR3 is considered “increased” when the expression level in a given patient is altered, relative to the basal expression level from the same patient by ≥40%; ≥45%; ≥50%; ≥55%; ≥60%; ≥65%; ≥ 70%; ≥75%; ≥80%; ≥85%; ≥90%; ≥95%; ≥100%; ≥105%; ≥110%; ≥115%; ≥120%; ≥125%; ≥130%; ≥135%; ≥140%; ≥145%; ≥150%;

In the following table, some suitable thresholds selected from the above are given

TABLE 4

| | | | | | dPCR or RT PCR | |
|---|---|---|---|---|---|---|
| | dPCR | | RT PCR | | not | |
| Gene | low | high | low | high | increased | increased |
| ErbB2 | <0.7 | ≥0.7 | >38.27 | ≤38.27 | <50% over | ≥50% over |
| FGFR2 | <0.01 | ≥0.01 | >47.50 | ≤47.50 | baseline | baseline |
| FGFR3 | <0.015 | ≥0.015 | >46.62 | ≤46.62 | | |

Surgery may involve, inter alia, TURBT (transurethral resection of bladder tumor), TURB (transurethral resection of bladder), and invasive bladder surgery.

Intravesical instillation therapy is oftentimes administered after TURBT an comprises direct administration of active therapeutics into the d, rather than systemic administration, Such therapy may involve the administration of BCG, as well as mitomycin C (MMC), epirubicin, pirarubicin and gemcitabine or other chemotherapeutic drugs, as well as anti-Her2 therapy.

Bacillus Calmette-Guerin or BCG is the most common intravesical immunotherapy for treating early-stage bladder cancer. It's used to help keep the cancer from growing and to help keep it from coming back. BCG is a germ that's related to the one that causes tuberculosis (TB), but it doesn't usually cause serious disease.

Monitoring means that the patient should undergo regular screening to determine ErbB2, FGFR2 and FGFR3 expression levels.

Systemic chemotherapy is oftentimes administered in advanced bladder cancer, both muscle-invasive localized disease and metastatic disease. Cisplatin-based multi-agent chemotherapy remains the cornerstone for systemic therapy. MVAC (methotrexate-vinblastine-doxorubicin-cisplatin) has been most rigorously studied, both neoadjuvantly and for palliation of metastatic disease. For metastatic disease, cisplatin-gemcitabine (GC) has compared favorably to MVAC due to improved tolerability with similar efficacy. GC has been adopted as standard therapy.

Anti-Her2 therapies include, inter alia, anti-Her2 antibodies, bi- or multispecific antibodies comprising a Her2 binding entity and at least one other entity, like e.g. an immunomodulator like an anti CD3 antibody or inflammatory cytokine, and anti-Her2 antibody drug conjugates, as shown in the following table.

TABLE 5
Examples for Anti-Her2 targeted therapies

| Drug name | Type | Manufacturer |
|---|---|---|
| Trastuzumab | Antibody | Roche |
| Trastuzumab | Antibody drug | Roche |
| emsantine | conjugate | |
| Pertuzumab | Antibody | Roche |
| Ertumaxomab | Multispecifc anti- | Trion/Fresenius |
| | Her2 × anti CD3 | |
| Margetuximab | Antibody | MacroGenics |
| Trastuzumab | Antibody drug | Daiichi Sankyo |
| deruxtecan | conjugate | |

FGFR inhibitors interfere with FGFR signaling, and hence provide different modes of affecting tumor survival. They allow for the increase of tumor sensitivity to regular anticancer drugs such as paclitaxel, and etoposide in human cancer cells and thereby enhancing antiapoptotic potential. Moreover, FGF signaling inhibition dramatically reduces revascularization, hitting upon one of the hallmarks of cancers, angiogenesis, and reduces tumor burden in human tumors that depend on autocrine FGF signaling based on FGF2 upregulation following the common VEGFR-2 therapy for breast cancer. In such a way, FGFR inhibitors can act synergistically with therapies to cut off cancer clonal resurgence by eliminating potential pathways of future relapse.

In addition, FGFR inhibitors might be effective on relapsed tumors because of the clonal evolution of an FGFR-activated minor subpopulation after therapy targeted to EGFRs or VEGFRs. Because there are multiple mechanisms of action for FGFR inhibitors to overcome drug resistance in human cancer, FGFR-targeted therapy is a promising strategy for the treatment of refractory cancer.

According to one or more embodiments, the FGFR inhibitor is an FGFR tyrosine kinase inhibitor. A tyrosine kinase inhibitor (TKI) is a drug that inhibits tyrosine kinases. Tyrosine kinases are enzymes responsible for the activation of many proteins by signal transduction cascades. Usually, they form the intracellular part of a transmembrane receptor, and, are activated upon extracellular ligand binding. Tyrosine kinases activate proteins by adding a phosphate group to the protein (phosphorylation), a step that TKIs inhibit. TKIs are typically used as anticancer drugs. For example, they have substantially improved outcomes in chronic myelogenous leukemia.

According to one or more embodiments, the FGFR inhibitor is at least one selected from the group as set forth in the following table.

TABLE 6
FGFR inhibitors

| Target | Drug name | Type | Manufacturer |
|---|---|---|---|
| Pan FGFR | Erdafitinib | small molecule | Janssen |
| Pan FGFR | Rogaratinib | small molecule | Bayer |
| FGFR1, FGFR2, | INCB054828/ | small molecule | Incyte |
| FGFR3 | Pemigatinib | | |
| FGFR1, FGFR2, | AZD4547 | small molecule | AstraZeneca |
| FGFR3 | | | |
| Pan FGFR | Derazantinib | small molecule | ArQule |
| | (ARQ 087) | | |
| FGFR1, FGFR2, | Infigratinib/ | small molecule | QED |
| FGFR3 | BGJ398 | | Therapeutics |
| Pan FGFR | JNJ-42756493 | small molecule | Johnson & |
| | | | Johnson |
| FGFR1, FGFR2, | Debio1347 | small molecule | Debiopharm |
| FGFR3 | | | |
| Pan FGFR | TAS-120 | small molecule | Taiho Oncology |
| FGFR1, FGFR2, | Dovitinib | small molecule | Novartis |
| FGFR3, VEGFR1, | (TKI258) | | |
| VEGFR2, VEGFR3, | | | |
| PDGFRb, KIT, | | | |
| RET, FLT3 | | | |
| FGFR1, FGFR2, | Lucitanib | small molecule | Clovis Oncology |
| VEGFR1, VEGFR2, | (E-3810) | | |
| VEGFR3 | | | |
| FGFR1, FGFR2, | Nintedanib | small molecule | Boehringer |
| FGFR3, VEGFR1, | | | Ingelheim |
| VEGFR2, VEGFR3, | | | |
| PDGFRa, PDGFRb, | | | |
| FLT3 | | | |
| FGFR1, FGFR2, | Infigratinib/ | small molecule | Novartis |
| FGFR3 | BGJ398 | | |
| Pan FGFR | LY2874455 | small molecule | Eli Lilly |
| FGFR1, VEGFR2, | Ponatinib | small molecule | Ariad |
| PDGFRa, | | | Pharmaceuticals |
| BCR-ABL, | | | |
| PDGFRb, c-SRC | | | |

Pan FGFR = FGFR1, FGFR2, FGFR3 and FGFR4

According to one embodiment, the sample is treated with silica- or germanium-coated magnetic particles, or with germanium beads or silica beads, and a chaotropic salt, for purification of the nucleic acids contained in said sample prior to the determination in step a).

Such methods using Germanium coated beads are for example disclosed in WO2013021027, the content of which is incorporated herein by reference. Kits utilizing this technology are for example marketed by STRATIFYER Molecular Pathology GmbH (“Magix Beads”). Such methods using silica coated beads are for example described by Boom et al (1990), the content of which is incorporated herein by reference. Kits utilizing this technology are for example marketed by BioMerieux, Qiagen or Promega.

According to another aspect, a kit is provided, comprising
a) at least one oligonucleotide comprising at least one nucleotide sequence which is capable of hybridizing to
   a nucleic acid molecule that encodes for any one of FGFR2, FGFR3, or ErbB2 or,
   an mRNA that encodes for any one of FGFR2, FGFR3, or ErbB2
which oligonucleotide is selected from the group consisting of
   an amplification primer (forward and/or reverse)
      a labelled probe, and/or
a substrate bound probe, and
b) one or more agents and/or devices suitable to isolate RNA from a liquid sample.

According to one embodiment, the one or more agents and/or devices suitable to isolate RNA from a liquid sample are agents and/or devices suitable to isolating exosomal RNA.

As discussed elsewhere herein, such agents and/or devices may comprise at least one of
agents suitable to bind water molecules, such as e.g. polyethylene glycol (PEG)
targeted immunomagnetic beads coated with any one of CD9, CD63, CD81m and/or
affinity membranes in a spin column format.

According to one embodiment, the kit comprises a set of forward/reverse primers capable of hybridizing to a nucleic acid molecule that encodes for FGFR2 FGFR3 and/or ErbB2, plus optionally a suitable probe.

According to one embodiment, the kit further comprises a set of forward/reverse primers plus optionally a suitable probe to a nucleic acid molecule that encodes for a reference gene. Suitable references genes are shown in the above table.

According to one embodiment, the kit further comprises a labelled probe that is labelled with one or more fluorescent molecules, luminescent molecules, radioactive molecules, enzymatic molecules and/or quenching molecules.

According to another aspect, the use of a kit according to any one of the aforementioned claims in a method of classifying a sample of a patient who suffers from or is at risk of developing urothelial or bladder cancer into one of at least two classifications.

### Examples

While the invention has been illustrated and described in detail in the drawings and foregoing description, such illustration and description are to be considered illustrative or exemplary and not restrictive; the invention is not limited to the disclosed embodiments. Other variations to the disclosed embodiments can be understood and effected by those skilled in the art in practicing the claimed invention, from a study of the drawings, the disclosure, and the appended claims. In the claims, the word “comprising” does not exclude other elements or steps, and the indefinite article “a” or “an” does not exclude a plurality. The mere fact that certain measures are recited in mutually different dependent claims does not indicate that a combination of these measures cannot be used to advantage. Any reference signs in the claims should not be construed as limiting the scope.

All amino acid sequences disclosed herein are shown from N-terminus to C-terminus; all nucleic acid sequences disclosed herein are shown 5′->3′.

### Materials and Methods

For this pilot study paraffin fixed pretreatment tissue samples from the first TURB of 74 pts participating in the BRIDGister Real World Experience trial and matched urine samples were prospectively collected and analyzed. RNA from FFPE tissues were extracted by commercial kits and analyzed by Therascreen FGFR IVD kit (Qiagen GmbH, Hilden) and BladderTyper (STRATIFYER Molecular Pathology GmbH, Cologne). In addition, urine samples were used for central isolation of extracellular vesicles and extraction of RNA and subsequently centrally analyzed for ACTB, CALM2, FGFR2, FGFR3 and ERBB2 by QIAcuity digital PCR (Qiagen, Hilden). A suitable kit was used for that purpose, like e.g. the exoEasy Kit (Qiagen, Hilden) und the miRCURY Exosome Kit Kit (Qiagen, Hilden). Exosomal RNA extraction resulted in valid gene expression results for 54 urine samples. 54 samples Associations studies based on Spearman and Kruskal-Wallis, MannWhitney and Sensitivity/Specificity tests were analyzed by JMP 9.0.0 (SAS software).

### Experimental Procedure

Exosomal RNA samples extracted from human urine were characterized for differences in their expression of potential bladder-cancer related genes. The expression levels of the selected target genes ACTB, CALM2, ERBB2, FGFR2 and FGFR3 were analyzed via digital PCR (dPCR) on the QIAcuity. Primer and probe oligo sequences and fluorophore of the probe for the house keeping gene ACTB is identical to the one in the therascreen FGFR RGQ RT-PCR kit (QIAGEN, cat.nr.: 874721). The 4 novel assay designs for CALM2, ERBB2, FGFR2 and FGFR3 are summarized in the below table 8.

For dPCR cDNA was synthesized from the RNA samples using the reverse transcription (RT) components of the therascreen FGFR RGQ RT-PCR kit including the RNA positive control. In addition universal human reference RNA (Thermo Fisher, cat.nr.: QS0639) was used as a positive control (table 9). RT procedures followed the manufactures recommendations and are summarized in table 10.

cDNA samples were further used in a dPCR reaction for absolute quantification of target molecules following the optimized procedures and configurations listed in Tables 11-13. Individual assays were run either in single plex with one assay per reaction or in multiplex with up to 4 assays per reaction. For each reaction 2-3 technical replicates were run. Individual absolute concentrations of target molecules (copies/μl) were calculated by the the QIAcuity Suite Software applying the autothreshold function to distinguish partitions with positive signal from negative ones.

### Results

### Correlation Between the Different Genes, and Between Same Genes in Tissue Vs Urine Sample

Relative gene expression of ERBB2, FGFR2 and FGFR3 from exosomal nucleic acid extraction from pre TURB urine and from bladder tissue samples was determined by normalization to ACTB and CALM2 (and compared to determination of respective gene expression using identical primer sequences for analysis of matched TURB tissue specimen).

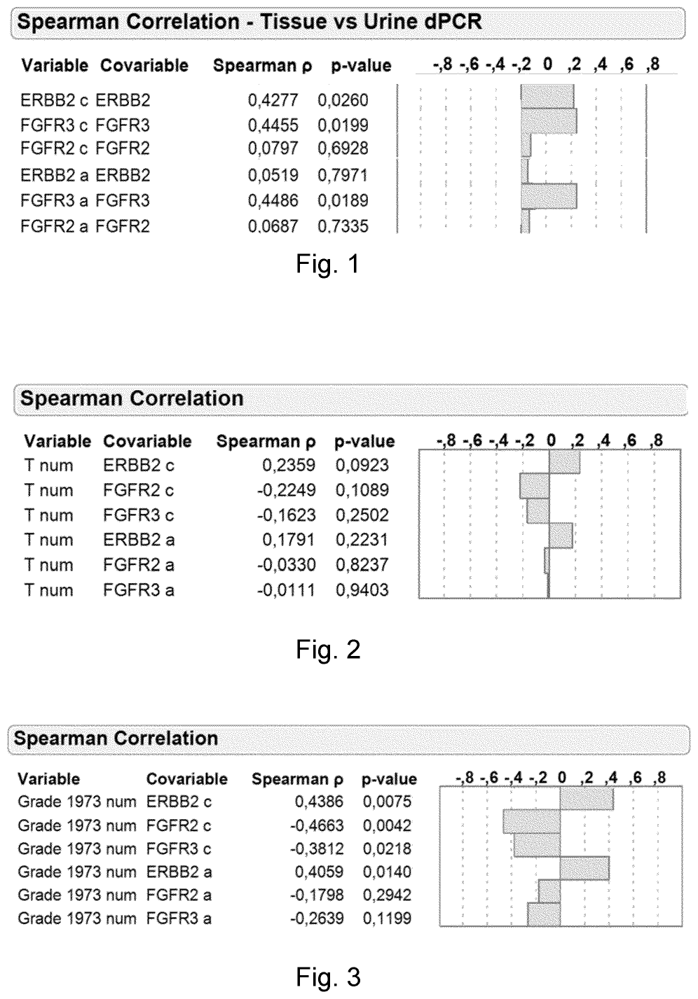

FIG. 1 shows spearman correlation of relative gene expression determined by dPCR on QIAcuity for ERBB2, FGFR2 and FGFR3 from urine exosomal RNA samples and matched FFPE tissues from bladder biopsies. “c” indicates gene expression values from urine after CALM2 normalization (Calmodulin 2 housekeeper). “a” indicates gene expression values from urine after ACTB normalization (Actin Beta housekeeper).

The figure shows that for all three genes, there is a high correlation between the gene expression as determined in by urine exosome analysis and FFPE tissue analysis. The data hence show that urine exosomal RNA samples can be taken as a surrogate for tissue samples from bladder biopsies. This opens up the pathway for a patient compliant, zero invasive screening approach that allows close patient monitoring at regular intervals.

In a further study, the correlation of gene expression in urine samples with tumor stage was analyzed.

FIG. 2 shows spearman correlation of relative gene expression determined by dPCR on QIAcuity for ERBB2, FGFR2 and FGFR3 from urine with tumor stage. “c” indicates gene expression values from urine after CALM2 normalization. “a” indicates gene expression values from urine after ACTB normalization.

For the tumor stage, score values (“T num”) have been assigned to the different tumor stages (see below description to the TNM system) according to the following table:

TABLE 7
Tumor stages

| Tumor stage | Score for correlation analysis |
|---|---|
| (TNM) | (T num) |
| TA | 0.5 |
| T1 | 1 |
| T2 | 2 |
| T3 | 3 |
| T4 | 4 |

It can be seen that expression of ERBB2 (Her-2/neu) is correlated with higher tumor stages, while expression of FGFR2 and/or FGFR3 is associated with lower tumor stages. This shows that when using CALM2 or ACTB as a reference gene/housekeeper, urine samples can be used for determining tumor stage, in particular when measuring the expression level of ERBB2 and/or FGFR2 or FGFR3.

In a further experiment, the correlation of gene expression in urine samples with WHO Grade 1973 (Chen et al., 2012; Mostofi et al. 1973) was analyzed. The World Health Organization 1973 classification system is an important prognosticator in bladder cancer. It is a 3 tier grading system G1-G3) endorsed by the European Association of Urology.

FIG. 3 shows spearman correlation of relative gene expression determined by dPCR on QIAcuity for ERBB2, FGFR2 and FGFR3 from urine exosome samples with tumor grade. WHO grade 1973 is positively associated with ERBB2 mRNA expression as determined from exosomal RNA that was prepared from urine samples, while FGFR2 and FGFR3 were negatively associated when CALM2 was used as reference gene. Normalization to ACTB also revealed positive association of ERBB2 with tumor grading. Hence, expression of ERBB2 (Her-2/neu) is associated with higher risk tumors, while expression of FGFR2 and/or FGFR3 is associated with lower risk tumors.

This indicates for the first time that tumor specific transcripts from urine detected by dPCR are associated with tumor characteristics of clinical value. In view of the limitations of subjective histopathological assessment and the fact that these molecular determinations were done by non-invasive methods from urine offers multiple advantages over current tissue based standards and adds independent information for tumor detection, tumor characterization as well as resulting prognostic and potentially predictive information.

TABLE 8
Summary of invented and predesigned assays used for this report. Oligos were
synthesized and ordered from Integrated DNA Technologies, Inc. (IDT).

| | | | Fwd | | | SEQ |
|---|---|---|---|---|---|---|
| | Target | Assay | Primer | Rev Primer | Probe | ID |
| ID | (Ensembl ID) | Reference | Sequence | sequence | Sequence | NO |
| ACTB* | Actin Beta | predesign | na | na | na | |
| | (ENSG00000075624) | | | | | |
| CALM2 | Calmodulin 2 | this | GAGCGA | AGTCAGTT | /5FAM/TCGCG | 1 - 3 |
| | (ENSG00000143933) | invention | GCTGAGT | GGTCAGCC | TCTCGGAAAC | |
| | | | GGTTGTG | ATGCT | CGGTAGC/3BHQ_ | |
| | | | | | 1/ | |
| ERBB2 | Erb-B2 | this | TCTGGAC | CCTGCTCC | /5HEX/AGGCC | 4 - 6 |
| | Receptor | invention | GTGCCAG | CTGAGGAC | AAGTCCGCAG | |
| | Tyrosine | | TGTGAA | ACAT | AAGCCCT/3BHQ_ | |
| | Kinase 2 | | | | 1/ | |
| | (ENSG00000141736) | | | | | |
| FGFR2 | Fibroblast | this | AAGCAGG | CAGATGGG | /5ROX/AGGCT | 7 - 10 |
| | Growth | invention | AGCATCG | ACCACACT | ACAAGGTACG | |
| | Factor | | CATTG | TTCCA | AAACCAGCAC | |
| | Receptor 2 | | | | TGG/3BHQ_2/ | |
| | (ENSG00000066468) | | | | | |
| FGFR3 | Fibroblast | this | AGCGCGT | CTCCCCGT | /5CY5/AGTGTG | 11 - 12 |
| | Growth | invention | ACTGTGC | CTTCGTCA | CGGGTGACAG | |
| | Factor | | CACTT | TCTC | ACGCTCC/3BHQ_ | |
| | Receptor 3 | | | | 2/ | |
| | (ENSG00000068078) | | | | | |

*from therascreen FGFR RGQ RT-PCR kit (QIAGEN, cat.nr.: 874721)

TABLE 9
Summary of methods and materials used in dPCR experiments

| Method | Materials |
|---|---|
| cDNA | Quantitect Reverse Transcription (RT) components from the |
| synthesis | therascreen FGFR RGQ RT-PCR kit (QIAGEN, cat. nr.: |
| | 874721) |
| | Positive control RNA (PC diluent) from the therascreen |
| | FGFR RGQ RT-PCR kit (QIAGEN, cat. nr.: 874721) |
| | Universal Human Reference RNA (Thermo Fisher, cat. nr.: |
| | QS0639) |
| dPCR | QIAcuity Four and Eight Platform System |
| | QIAcuity dPCR Probe Mastermix (QIAGEN, cat. nr.: 250101) |
| | 8.5k 96-well nanoplate (QIAGEN, cat. nr.: 250021) |
| | Dilution buffer (RNAse-free H₂O) |
| | QIAcuity Suite Software version 1.2.18 |

TABLE 10
Summary of Reverse Transcription reaction setup.

cDNA synthesis using the RT components of the therascreen ® FGFR RGQ RT-PCR Kit
RNA input: Urinary exoRNA 14 μL each in 1.5 mL tubes, each extracted from 20 mL urine
Control RNA: Positive Control and dilution buffer (diluent) from the therascreen FGFR
RGQ RT-PCR kit
Procedure:
1. Cool down rack
2. Let all RT components defreeze at room temperature except PC and PC diluent that
are defreezed at 0-8° C. for 30-60 min
3. Prepare positive control RNA samples and exoRNA samples on ice.
4. Vortex RT Mastermix components 3x for 3 sec and centrifuge.
5. Prepare RT Mastermix on ice

| | |
|---|---|
| 6. Vortex samples and RT MM 3x for 3 sec and centrifuge | store on ice |

7. Combine Samples and RT MM and run RT Cycling

| | Lot number | μl/reaction | |
|---|---|---|---|
| Reverse transcription | | | |
| reaction mix | | | |
| RT Buffer 1 | 166016630 | 5 | |
| RT Buffer 2 | 166017369 | 2.5 | |
| RT Primer Mix | 166016536 | 1.25 | |
| RT | 166016535 | 1.25 | |
| | Total | 10 | |
| Positive Control Sample (PC): | | | |
| PC | 166012745 | 5 | prepare on ice |
| PC Diluent | 166012746 | 10 | |
| | Total | 15 | |

| | RNAs | PC |
|---|---|---|
| RT Reaction: | | |

| | | |
|---|---|---|
| Template | 14 μl (+1 μl | 15 μl |
| | H₂O) | |

| | | | |
|---|---|---|---|
| Reverse transcription reaction mix | 10 | μl | 10 μl |
| RT Cycling: | | | |
| 20° C. | 15 | min | |
| 42° C. | 30 | min | |
| 95° C. | 3 | min | |

TABLE 11
Overview of dPCR reaction setup

| Reagent | 1x | | final concentration |
|---|---|---|---|
| QN Probe PCR MM 4x | 3.0 | μl | 1x |
| PrimerProbeMix Assay 1 | 0.6 | μl | 0.8 μM Primer |
| (20x) | | | and 0.4 μM Probe |
| PrimerProbeMix Assay 2 | 0.6 | μl | for each mix |
| (20x) | | | |
| PrimerProbeMix Assay 3 | 0.6 | μl | |
| (20x) | | | |
| PrimerProbeMix Assay 4 | 0.6 | μl | |
| (20x) | | | |
| Water | Add to 12 μl | | |
| Template | 0.5-1.5 | μl | |
| | 12 | μl | |

TABLE 12
Overview of dPCR cycling conditions

| Temperature | Duration in min | Cycles |
|---|---|---|
| 95° C. | 02:00 | 1x |
| 95° C. | 00:15 | 40x |
| 60° C. | 00:30 | |
| 45° C. | 01:00 | 1x |

TABLE 13
Overview of QIAcuity instrument settings
Priming Profile

| | |
|---|---|
| PrimeTravelDistance | 2.6 |
| Velocity (mm/sec) | 4 |

Imaging settings

| Channel | Exposure time (ms) | Gain |
|---|---|---|
| GREEN | 500 | 6 |
| YELLOW | 500 | 6 |
| ORANGE | 400 | 6 |
| RED | 300 | 4 |
| CRIMSON | 400 | 8 |

### REFERENCES

Li P, Kaslan M, Lee S H, Yao J, Gao Z. Progress in Exosome Isolation Techniques. Theranostics. 2017; 7(3): 789-804. Published 2017 Jan. 26. doi:10.7150/thno.18133
Boom et al (1990), J Clin Microbiol. 1990 March; 28(3): 495-503
Chen Z et al. PLOS One. 2012; 7(10): e47199. doi: 10.1371/journal.pone.0047199. Epub 2012 Oct. 17. PMID: 23082147; PMCID: PMC3474808.
Kozera and Rapacz, Reference genes in real-time PCR, J Appl Genet. 2013; 54(4): 391-406
Mostofi, F. et al (1973). Histological typing of urinary bladder tumours. World Health Organization.

### SEQUENCES

The following sequences form part of the disclosure of the present application. A WIPO ST 25 compatible electronic sequence listing is provided with this application, too. For the avoidance of doubt, if discrepancies exist between the sequences in the following table and the electronic sequence listing, the sequences in this table shall be deemed to be the correct ones.

| SEQ | Qualiier | Sequence |
|---|---|---|
| 1 | CALM2 Fwd Primer Sequence | GAGCGAGCTGAGTGGTTGTG |
| 2 | CALM2 Rev Primer sequence | AGTCAGTTGGTCAGCCATGCT |
| 3 | CALM2 Probe Sequence | TCGCGTCTCGGAAACCGGTAGC |
| 4 | ERBB2 Fwd Primer Sequence | TCTGGACGTGCCAGTGTGAA |
| 5 | ERBB2 Rev Primer sequence | CCTGCTCCCTGAGGACACAT |
| 6 | ERBB2 Probe Sequence | AGGCCAAGTCCGCAGAAGCCCT |
| 7 | FGFR2 Fwd Primer Sequence | AAGCAGGAGCATCGCATTG |
| 8 | FGFR2 Rev Primer sequence | CAGATGGGACCACACTTTCCA |
| 9 | FGFR2 Probe Sequence | AGGCTACAAGGTACGAAACCAGCACTGG |
| 10 | FGFR3 Fwd Primer Sequence | AGCGCGTACTGTGCCACTT |
| 11 | FGFR3 Rev Primer sequence | CTCCCCGTCTTCGTCATCTC |
| 12 | FGFR3 Probe Sequence | AGTGTGCGGGTGACAGACGCTCC |

As used herein, the terms “general,” “generally,” and “approximately” are intended to account for the inherent degree of variance and imprecision that is often attributed to, and often accompanies, any design and manufacturing process, including engineering tolerances, and without deviation from the relevant functionality and intended outcome, such that mathematical precision and exactitude is not implied and, in some instances, is not possible.

All the features and advantages, including structural details, spatial arrangements and method steps, which follow from the claims, the description and the drawing can be fundamental to the invention both on their own and in different combinations. It is to be understood that the foregoing is a description of one or more preferred exemplary embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

1. A method of classifying a patient that suffers from or is at risk of developing urothelial or bladder cancer, said method comprising the steps of:
a) taking a urine sample from the patient,
b) extracting RNA from said urine sample, and
c) determining in said RNA, the expression level of at least one gene encoding for a receptor selected from the group consisting of FGFR2, FGFR3 or ErbB2,
d) classifying said urine sample of the patient from the outcome of step c) into one of at least two classifications,
wherein the one or more expression level(s) determined in step c) are normalized with the expression level of the housekeeping gene CALM2 to obtain one or more normalized expression level(s).

2. The method according to claim 1, wherein the RNA is extracted from exosomes in said urine sample.

3. The method according to claim 1, wherein, in step c) the expression level of at least ErbB2 is determined.

4. The method according to claim 1, wherein, in step c) the expression level of at least
ErbB2 and
FGFR2 and/or FGFR3
is determined.

5. (canceled)

6. The method according to claim 1, wherein said expression level(s) is/are determined by at least one of
(i) a hybridization-based method, in which labeled, single stranded probes are used
(ii) a PCR based method, which method comprises a polymerase chain reaction (PCR)
(iii) a method based on the electrochemical detection of particular molecules, which method encompasses an electrode system to which molecules bind under creation of a detectable signal,
(iv) an array based method, which comprises the use of a m microarray and/or biochip, and/or
(v) an immunological method, in which one or more target-specific protein binders are used.

7. The method according to claim 6, wherein the PCR based method is digital PCR (dPCR)

8. (canceled)

9. (canceled)

10. (canceled)

11. The method according to claim 1, wherein the step d) of classifying said urine sample of the patient from the outcome of step c) into one of at least two classifications comprises a classification into at least one of
urothelial or bladder cancer negative or positive
low risk or high risk urothelial or bladder cancer

12. The method according to claim 1, wherein said urine sample is treated with silica- or germanium-coated magnetic particles, or with germanium beads or silica beads, and a chaotropic salt, for purification of the nucleic acids contained in said urine sample prior to the determination in step a).


SEQUENCE LISTING (12 sequences)

SEQ ID NO: 1 - DNA, 20 bp, synthetic construct
  misc_feature 1..20: note = primer sequence
  source 1..20: mol_type = other DNA
gagcgagctgagtggttgtg

SEQ ID NO: 2 - DNA, 21 bp, synthetic construct
  misc_feature 1..21: note = Primer sequence
  source 1..21: mol_type = other DNA
agtcagttggtcagccatgct

SEQ ID NO: 3 - DNA, 22 bp, synthetic construct
  misc_feature 1..22: note = Primer sequence
  source 1..22: mol_type = other DNA
tcgcgtctcggaaaccggtagc

SEQ ID NO: 4 - DNA, 20 bp, synthetic construct
  misc_feature 1..20: note = Primer sequence
  source 1..20: mol_type = other DNA
tctggacgtgccagtgtgaa

SEQ ID NO: 5 - DNA, 20 bp, synthetic construct
  misc_feature 1..20: note = Primer sequence
  source 1..20: mol_type = other DNA
cctgctccctgaggacacat

SEQ ID NO: 6 - DNA, 22 bp, synthetic construct
  misc_feature 1..22: note = Primer sequence
  source 1..22: mol_type = other DNA
aggccaagtccgcagaagccct

SEQ ID NO: 7 - DNA, 19 bp, synthetic construct
  misc_feature 1..19: note = Primer sequence
  source 1..19: mol_type = other DNA
aagcaggagcatcgcattg

SEQ ID NO: 8 - DNA, 21 bp, synthetic construct
  misc_feature 1..21: note = Primer sequence
  source 1..21: mol_type = other DNA
cagatgggaccacactttcca

SEQ ID NO: 9 - DNA, 28 bp, synthetic construct
  misc_feature 1..28: note = Primer sequence
  source 1..28: mol_type = other DNA
aggctacaaggtacgaaaccagcactgg

SEQ ID NO: 10 - DNA, 19 bp, synthetic construct
  misc_feature 1..19: note = Primer sequence
  source 1..19: mol_type = other DNA
agcgcgtactgtgccactt

SEQ ID NO: 11 - DNA, 20 bp, synthetic construct
  misc_feature 1..20: note = Primer sequence
  source 1..20: mol_type = other DNA
ctccccgtcttcgtcatctc

SEQ ID NO: 12 - DNA, 23 bp, synthetic construct
  misc_feature 1..23: note = Primer sequence
  source 1..23: mol_type = other DNA
agtgtgcgggtgacagacgctcc